(19) United States
(12) Patent Application Publication
(10) Pub. No.: US 2007/0016001 A1
(43) Pub. Date: Jan. 18, 2007

(54) Method and apparatus for multi-exposure medical examination of a subject with automatic control of the patient table

(76) Inventor: Gudrun Graf, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/418,654

(22) Filed: May 5, 2006

(30) Foreign Application Priority Data
May 6, 2005 (DE) 10 2005 021 065.1

(51) Int. Cl. A61B 5/05

(52) U.S. Cl. 600/410

(57) ABSTRACT

In a medical examination device and method for the generation of examination images of an object, at least one setting value relevant for the examination can be set with at least one stored or manually entered item of the object specific and/or examination-specific information, and a table for support of the object to be examined is moved by a control system and in the course of the examination several examination images of adjacent or adjoining regions of the object are generated when the table is moved. The movement of the table is controlled by the control system with the assistance of the object-specific and or examination-specific information.

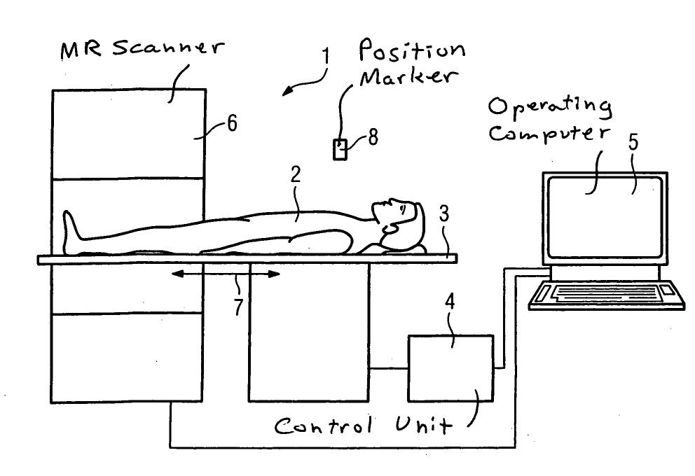

### BACKGROUND OF THE INVENTION

1. Field of the Invention

The present invention relates to a medical examination device for the generation of examination images of an object of the type wherein at least one value relevant for the examination can be set by entered or stored object-specific and/or examination-specific information.

2. Description of the Prior Art

A method for the generation of magnetic resonance images of an object as well as a corresponding magnetic resonance device are known from US 2002/0087066 A1, wherein object-specific and/or examination-specific parameters are supplied to the magnetic resonance device. The magnetic resonance device determines optimal settings and/or setting ranges of sequence parameters for a combination of the supplied object-specific and/or examination-specific parameters for the generation of magnetic resonance images. For example the size, the mass or the length of an object can be used as object-specific parameters. The region of the object to be mapped can be, for example, used as an examination-specific parameter. The field of view can be used as a sequence parameter within the scope of the method.

From DE 103 35 037 A1 a control method for a medical device is known, in which movements can be performed with a pointing instrument, said images being recorded by an image detection system. With the assistance of the recorded information the medical device is controlled, for example a patient bed can be shifted accordingly.

From EP 1 362 550 A1 a method for the scanning of the entire body of a patient is known, wherein the patient table can be moved continuously. As a result, the generation of a large image formed by combining several individual images.

In US 2005/0074085 A1 a medical examination device is described in which the various scanning methods can be selected to assist in controlling shifting of the patient table.

An angiography method is known from WO 03/034901 A2 in which a total image is formed from several partially overlapping individual images.

Although these known medical examination devices already relieve the user from making individual settings, there is need for a medical examination device as a result of which the user is even further relieved.

### SUMMARY OF THE INVENTION

In a medical examination device of the type the above need is achieved in accordance with the invention by a table for supporting the object to be examined that can be moved by a control system and, in the course of the examination, generating multiple examination images of adjacent or adjoining regions of the object when the table is moved, with the movement of the table being controlled by the control system, of generating the multiple images, dependent on the object-specific and/or examination-specific information.

The invention is based on the recognition of the fact that the automatic setting of setting values relevant for the examination can also be applied to the table moving technology. Using the table motion technique an object, for example a patient, can be examined in sections. For this purpose the patient is placed on a movable table which is moved such that an examination image of the desired region is made. In the case of conventional examination devices the positioning of the table must be performed manually, with a certain difficulty. The individual procedure steps of the table and the optimum field of view of the examination device must be calculated and entered. This sequence is expensive and the danger exists that optimum settings will not be made for the examination device.

By contrast the moving of the table in the case of the inventive examination device is controlled with the object-specific and/or examination-specific information, so the setting and positioning of the table takes place automatically. In this way the optimum setting can be automatically determined.

In a preferred improvement of the invention, the size of the object to be examined can be entered by the user or can be automatically recorded as information for the control system. With this information the control system can control the movement of the table such that the various examination regions of the object are optimally aligned on the field of view of the examination device. In the case of large persons, for example, four examination images can be prepared, for which purpose the table is correspondingly shifted. In the case of small persons, for example, three examination images suffice. In the process the setting of the table movement and the positioning of the table occur automatically by the examination device.

In a further embodiment of the invention the extent of the overlapping of adjacent examination images can be entered by the user as information for the control system. In the case of a smaller overlapping the measurement can be made with a smaller field of view, so that the resolution is increased.

In another embodiment of the inventive medical examination device the start and end positions of the region to be examined can be entered by the user, from which the movement of the table is controlled by the control system. The examination region is defined by the entry of the start and positions, the subsequent adaptation of the protocol takes place automatically.

To increase user friendliness in a further embodiment of the inventive medical examination device the distance between the start and end positions can be entered by a user with a light marker or manually and that the movement of the table is controlled by the control system dependent on the input distance. Using the light marker an especially convenient setting of the start and end positions is possible, and the setting can be visually inspected immediately.

In a further embodiment the case of the inventive medical examination device the multiple examination images can be combined into a complete image. In the process the position, the overlapping and the field of view of the individual examination images are automatically determined with the entered or recorded object-specific and/or examination-specific information.

In addition, the invention relates to a method for the generation of examination images of an object with a medical examination device in which at least one setting value relevant for the examination is determined with at least one item of object-specific and/or examination-specific information that is entered by a user or automatically recorded.

In the case of the inventive method the examination device is a table that can be moved by a control system for support of the object to be examined and that in the course of the examination several examination images of adjacent or adjoining regions of the object are generated by moving the table and the movement of the table is controlled by the control system with object-specific and/or examination-specific information.

In a further embodiment of the inventive method provision can be made that the size of the object to be examined can be entered by the user as information for the control system or is automatically recorded. Alternatively or additionally the extent of the overlapping of adjacent examination images can be entered by the user as information for the control system. In addition, the start and end positions of the region to be examined can be entered by the user, with which the movement of the table is controlled by the control system.

### DESCRIPTION OF THE DRAWING

The single FIGURE is a schematic block diagram of an inventive medical examination device operable according to the inventive method.

### DESCRIPTION OF THE PREFERRED EMBODIMENTS

The medical examination device is shown as a magnetic resonance device 1 and has a basic field magnet system for the generation of a basic magnetic field and a gradient coil system for the generation of gradient fields. The structure and function of such a magnetic resonance device is known and does not require further explanation.

A patient 2 is positioned on a table 3, which serves as a horizontally movable support device. The movement and positioning of the table 3 take place via a control unit 4. A display and operating computer 5 is connected both to the control unit 4 as well as to the basic field magnet system and the gradient coil system, which together form an MR scanner 6 (data acquisition system).

For conducting the examination, the patient 2 lying on the table 3 is moved in the direction indicated by the double arrow 7 so that the respective region of the patient 2 to be examined is located in the MR scanner 6. After the generation of an examination image the table 3 is moved by the control unit 4 until a further region of the patient 2 to be examined is located within the MR scanner 6. In this way, three or four individual images are generated, which are subsequently joined into a whole image, for example a whole body exposure or a an exposure of the entire spinal cord.

In the registration of the patient, the size of the patient is entered by a user at the display and operation device. This size is a setting value relevant for the examination and serves the purpose of automatically determining examination-specific information. In this case the examination protocol in which the precise sequence of an examination is set is adapted from the entered size of the patient. This can take place before a first diagnostic measurement. The user can manually set the starting point of the examination. In the process the user has the option to decide for a greater overlapping or to measure the individual examination images with constant overlapping with a smaller field of view. In this case a higher resolution of the individual images results. In the case of smaller patients it is recommended that the user, instead of a four-step measurement, in which four individual images are generated, conduct a three-step measurement at the same resolution. If a more far-reaching automation is desired, the decision as to whether three or four examination images will be obtained can be made by the control unit 4, which moves the table 3 correspondingly in the subsequent examination.

The magnetic resonance device 1 can also be used so that first the start position is entered by a position marker 8 schematically represented in the FIGURE; subsequently the end position of the region of the patient to be examined is entered. The distance between the start and end positions determines the examination region. This data serves as examination-specific information, with which the control unit 4 correspondingly controls the movement of the table 3.

If a whole body measurement for screening is performed with a person having a height of 1.85 meters, four steps with a 500 mm field of view are needed, thus four examination images, with some overlapping. If the same measurement is performed with smaller persons, for example on a patient with a body size of about 1.60 meters, this could also be done in four steps, with either the distortions in the marginal areas being reduced by greater overlapping of the individual partial sections, or a higher resolution being achieved by reducing the field of view to 420 mm.

Another example is a spinal cord screening. By means of a light beam localizer marking in the head region and in the pelvic region, the cranial end of the spinal cord as well as the distal end of the spinal cord can be determined. The input of the distance between the upper and lower light beam localizer can be either pre-defined and then entered or an automatic determination of this length can take place prior to the screening process. In any event the distance between the position markers must be first determined and entered manually or automatically into the device as examination-specific parameters so that the suitable image sequence including the adaptation of the parameters can be automatically set. By means of the automation, a two-step measurement can be made in the case of small persons and in the case of large persons a three-step measurement can be performed, since the region to be scanned is known in advance. This can also apply to partial sections of the spinal column, for example such as HWS-BWS, BWS-LWS or in the case of longer bones.

In the case of scanners with a smaller field of view in the Z-direction, the inventive device can be used particularly advantageously, since here individual examination images must be placed next to each other significantly more frequently and an optimal scan program is automatically created without the user having to calculate in advance how the program will be set up. The images are then combined in the operating computer S (which thus also serves as an imaging computer) in normal manner to show a whole body exposure or a total spinal cord exposure.

Although modifications and changes may be suggested by those skilled in the art, it is the intention of the inventor to embody within the patent warranted hereon all changes and modifications as reasonably and properly come within the scope of her contribution to the art.

I claim as my invention:

1. A medical examination system comprising:
a subject table adapted to receive a subject thereon;
a medical data acquisition device in which the subject is positionable by movement of said subject table, said medical data acquisition device being adapted to interact with the subject to obtain data therefrom representing an examination image; and
a control unit that operates said subject table and said medical data acquisition device, said control unit containing information selected from the group consisting of subject-specific information and examination-specific information, and said control unit operating said medical data acquisition device and said subject table to acquire data representing a plurality of examination images respectively from multiple regions of the subject while automatically moving said subject table dependent on said information.

2. A medical examination system as claimed in claim 1 comprising a user input connected to said control unit allowing manual entry of said information into said control unit.

3. A medical examination system as claimed in claim 1 wherein said control unit comprises a memory in which said information is stored.

4. A medical examination system as claimed in claim 1 wherein said information comprises examination-specific information indicating a degree of overlapping of said examination images.

5. A medical examination system as claimed in claim 1 wherein said information comprises examination-specific information indicating a start position and an end position of said subject table, that define an extent of said multiple regions of the subject from which said respective examination images are obtained.

6. A medical examination system as claimed in claim 5 wherein said information comprises a distance between said start position and said end position that defines said start position and said end position.

7. A medical examination system as claimed in claim 1 comprising an image computer that combines said plurality of examination images into a single, complete image.

8. A medical examination method comprising the steps of:
placing a subject on a subject table;
moving the subject on the subject table into a medical data acquisition device by movement of said subject table, and with said medical data acquisition device, obtaining data from the subject representing an examination image; and
automatically operating said subject table and said medical data acquisition device, with control unit containing information selected from the group consisting of subject-specific information and examination-specific information, said subject table to acquire data representing a plurality of examination images respectively from multiple regions of the subject while automatically moving said subject table dependent on said information.

9. A medical examination method as claimed in claim 8 comprising allowing manual entry of said information into said control unit.

10. A medical examination method as claimed in claim 8 comprising storing said information in a memory in said control unit.

11. A medical examination method as claimed in claim 8 comprising employing as said information, examination-specific information indicating a degree of overlapping of said examination images.

12. A medical examination method as claimed in claim 8 comprising employing as said information, examination-specific information indicating a start position and an end position of said subject table, that define an extent of said multiple regions of the subject from which said respective examination images are obtained.

13. A medical examination method as claimed in claim 12 comprising employing as said information, a distance between said start position and said end position that defines said start position and said end position.

14. A medical examination method as claimed in claim 8 comprising an electronically combining said plurality of examination images into a single, complete image.
